(19) United States
(12) Patent Application Publication
(10) Pub. No.: US 2026/0278890 A1
(43) Pub. Date: Sep. 17, 2026

(54) Recognition-Based Medical Imaging System: Methods and Systems for Enhanced Medical Image Acquisition and Reconstruction Using Recognition Physics

(71) Applicant: Jonathan Washburn, Austin, TX (US)

(72) Inventor: Jonathan Washburn, Austin, TX (US)

(21) Appl. No.: 19/565,367

(22) Filed: Mar. 12, 2026

Related U.S. Application Data
(63) Continuation-in-part of application No. 19/296,734, filed on Aug. 11, 2025.
(60) Provisional application No. 63/771,419, filed on Mar. 13, 2025.

(51) Int. Cl. G06T 12/10; A61B 6/00; A61B 6/03; A61B 8/00; G06T 7/11

(52) U.S. Cl. CPC G06T 12/10; A61B 6/037; A61B 6/5205; A61B 8/5207; G06T 7/11; G06T 2207/10072

(57) ABSTRACT

A recognition-based medical imaging framework is disclosed in which a recognition coverage value is computed from a quality signal using a recognition coverage function parameterized by a recognition scaling parameter, and the recognition coverage value is used to control one or more acquisition parameters during an imaging exam and to weight one or more reconstruction operations for image formation. In some embodiments, the framework is implemented in computed tomography, magnetic resonance imaging, ultrasound, positron emission tomography, or digital radiography systems, and may include recognition-based acquisition control, recognition-weighted reconstruction, voxel-level image enhancement, and diagnostic feature extraction. The framework may be executed on one or more processors associated with an imaging system and may output medical images and associated metadata identifying acquisition settings, recognition coverage information, and reconstruction settings.

### CROSS-REFERENCE TO RELATED APPLICATIONS

This application is a continuation-in-part of U.S. Nonprovisional patent application Ser. No. 19/296,734, filed August 11,2025, which claims the benefit of U.S. Provisional Patent Application No. 63/771419, filed Mar. 13, 2025, titled “Recognition-Based Medical Imaging System: Methods and Systems for Enhanced Medical Image Acquisition and Reconstruction Using Recognition Physics.”

### STATEMENT REGARDING FEDERALLY SPONSORED RESEARCH OR DEVELOPMENT

No federal funding or government contract supported the research or development disclosed in this application.

### NAMES OF THE PARTIES TO A JOINT RESEARCH AGREEMENT

Not applicable.

### REFERENCE TO A SEQUENCE LISTING, A COMPUTER PROGRAM LISTING, OR A LARGE TABLE APPENDIX

Not applicable.

### BACKGROUND OF THE INVENTION

Medical imaging systems—including magnetic resonance imaging (MRI), computed tomography (CT), ultrasound, positron emission tomography (PET), and digital radiography (DR/X-ray)—are central to modern diagnosis and therapy planning. Despite significant progress in detector technology, acquisition control, and reconstruction algorithms, these modalities still face persistent trade-offs among radiation or energy exposure, acquisition time, motion sensitivity, image noise, artifact burden, and computational latency.

In CT and DR/X-ray, image quality often improves with increased tube current or exposure time, but higher radiation dose elevates patient risk. Vendors employ automatic exposure control and tube-current/kVp modulation to mitigate dose, yet these methods are typically heuristic, scanner-specific, or anatomy-templated and may not adapt at sufficiently fine spatial or temporal scales to patient-specific variation, motion, or contrast distribution throughout an exam.

In MRI, sequence design (e.g., TR/TE/flip angle), sampling trajectories, and acceleration strategies (e.g., parallel imaging and compressed sensing) can reduce scan time and improve resolution. However, selecting these parameters remains a complex, multi-objective process influenced by hardware constraints, patient physiology, and pathology. Existing adaptive or model-based approaches often optimize for limited criteria or single sequences and may not generalize across anatomies, field strengths, and hardware vendors without extensive manual tuning.

In ultrasound, beamforming, time-gain compensation, adaptive focusing, and speckle-suppressing post-processing improve contrast and lesion conspicuity. Nevertheless, conventional pipelines can struggle to distinguish low-contrast structures in challenging acoustical environments, and dynamic adaptation is frequently constrained by device throughput, simplistic feedback metrics, or operator-dependent presets.

In PET, iterative reconstruction methods (e.g., OSEM variants with priors) and acquisition-time optimization aim to elevate signal-to-noise ratio (SNR) while limiting patient dose and scanner occupancy. These solutions can be sensitive to regularization choices, convergence tuning, and count statistics, and they may not leverage patient-specific informational structure available during acquisition to guide time allocation or reconstruction weighting.

Across modalities, advanced iterative and learning-based reconstructions (e.g., penalized likelihood, dictionary/transform sparsity, and deep-learning denoisers) can deliver material gains in noise-resolution trade-offs. However, such improvements are typically realized post-acquisition and may not feed back into the live acquisition loop to modulate energy delivery, trajectory selection, or sampling density in real time. Moreover, learned methods can be sensitive to dataset bias and require careful curation, calibration drift monitoring, and uncertainty handling to maintain clinical reliability.

A unifying limitation of current practice is the absence of a principled, modality-agnostic framework that: (i) quantifies the informational coherence present in measured data at the point of acquisition; (ii) uses that quantification to adapt acquisition parameters, sampling, and energy delivery in real time for the individual patient; and (iii) carries the same quantification consistently into reconstruction to guide weighting, regularization, and filtering. Existing pipelines frequently treat acquisition control and image reconstruction as loosely coupled stages, leaving potential performance on the table.

There is thus a need for systems and methods that provide a rigorous, quantifiable measure of information coverage or coherence that can be computed on-the-fly, is stable across anatomies and devices, and is simple enough to implement on embedded or accelerated hardware (e.g., FPGA/GPU) with tight latency budgets. Such a measure should drive both upstream acquisition control (dose/current/sequence/trajectory/beamforming) and downstream reconstruction (frequency-domain weighting, spatial regularization, and voxel-wise filtering) in a coordinated way.

Recognition Physics introduces a mathematically grounded approach in which an optimal informational scaling parameter (denoted herein as Xopt and, in certain embodiments, defined by the ratio phi over pi) and an associated recognition coverage function (denoted herein as Fcov) quantify the degree to which measured signals are informationally coherent relative to noise and modeling uncertainty. While specific formulae and implementations are provided elsewhere in this specification, the salient point for the background is that Xopt and Fcov furnish a compact, modality-agnostic control signal that can be evaluated rapidly and used consistently across acquisition and reconstruction.

In acquisition, a recognition-coverage-driven controller can, for example: (i) modulate tube current and kVp in CT/DR/X-ray; (ii) select or adapt MRI sequence parameters (e.g., TR/TE/flip angle) and k-space sampling density; (iii) adjust ultrasound beamforming weights and time-gain compensation; and (iv) allocate PET acquisition time or apply count-domain weighting. In reconstruction, the same coverage measure can guide frequency-space filters, spatially varying regularization, or voxel-wise weighting to improve SNR and resolution while preserving diagnostically important features.

Because Xopt and Fcov are defined by a small number of stable quantities, they can be computed under strict latency (sub-frame or sub-projection) constraints and deployed on practical accelerators. This enables closed-loop behavior in which intermediate coverage estimates inform immediate acquisition adjustments and then carry forward into the reconstruction stage without inconsistencies between what was measured and how it is processed.

The medical imaging industry—exceeding forty-five billion dollars annually—would benefit from a unified, recognition-based framework that (i) reduces radiation or energy exposure without sacrificing diagnostic confidence; (ii) improves resolution and contrast-to-noise in anatomies and pathologies that challenge existing heuristics; (iii) shortens or stabilizes scan time via smarter sampling; and (iv) integrates with existing scanner hardware and clinical workflows through well-defined interfaces.

The present disclosure addresses these needs by providing systems and methods that compute patient- and context-specific recognition coverage during acquisition and apply the same coverage consistently in reconstruction. By establishing a common informational currency across modality control and image formation, the disclosed technology advances beyond heuristic dose/parameter presets and after-the-fact denoising, enabling principled, real-time, closed-loop optimization that is compatible with current clinical infrastructure.

### SUMMARY OF THE INVENTION

The invention provides a recognition-based framework for medical imaging that unifies real-time acquisition control and image reconstruction through a common informational signal called recognition coverage. A recognition scaling parameter, Xopt (in certain embodiments set to the ratio phi over pi or determined by an optimization on historical or live data), and a recognition coverage function, Fcov(r, Xopt), quantify informational coherence of measured signals r and are computed rapidly during acquisition. The same Fcov is then used downstream in reconstruction to guide frequency-domain weighting, spatially varying regularization, and voxel-wise filtering, producing improved resolution and contrast-to-noise while enabling dose or energy reductions and maintaining clinical throughput.

In one aspect, a method comprises: (i) receiving raw acquisition data and associated scanner/patient metadata; (ii) computing one or more live quality metrics (including but not limited to signal-to-noise ratio, local contrast, count statistics, motion or artifact indicators) and deriving a recognition coverage value Fcov for the current context using Xopt; (iii) adaptively selecting or modulating acquisition parameters in a closed loop based on Fcov (for example, exposure settings, sequence timing, sampling density, beamforming, or dwell time); (iv) reconstructing images using the same Fcov to weight filters and regularizers in the projection, k-space, frequency, or image domain; and (v) outputting DICOM images and companion metadata that record acquisition and reconstruction settings and coverage statistics.

In another aspect, a system comprises: an imaging sensor assembly (for CT/DR, an X-ray tube and detector; for MRI, RF coils and gradients; for ultrasound, a transducer array; for PET, scintillation detectors); an acquisition controller configured to compute recognition coverage in real time and to drive modality-specific parameters; and a reconstruction engine configured to incorporate the same coverage values into reconstruction weighting. The controller and reconstruction engine may execute on CPU/GPU and/or FPGA hardware under sub-projection, sub-frame, or view-by-view latency constraints and communicate with scanner subsystems through vendor-supported APIs and safety interlocks.

In another aspect, a non-transitory computer-readable medium stores instructions that, when executed by one or more processors, cause the processors to perform the method of paragraph [0020], including computing Fcov(r, Xopt) and applying it both to acquisition control and to reconstruction weighting.

In certain embodiments, Fcov(r, Xopt) is defined as r divided by (r plus Xopt), providing a stable, bounded weight that increases with coherent signal and decreases with noise or uncertainty. In some embodiments, Xopt is set to phi divided by pi; in other embodiments, Xopt is estimated by maximizing an information criterion or fitted from calibration or prior studies. The framework is modality-agnostic and can accept alternative monotone coverage functions that preserve the closed-loop behavior described herein.

Modality-specific embodiments include: (i) CT/DR: view- or region-specific tube-current and kVp modulation driven by Fcov, and reconstruction with Fcov-weighted filters or iterative penalties; (ii) MRI: adaptive selection of TR/TE/flip angle and sampling density in k-space based on coverage maps, with reconstruction using Fcov-weighted parallel/iterative or compressed-sensing pipelines; (iii) ultrasound: dynamic beamforming, time-gain compensation, and speckle-aware post-processing guided by coverage, with reconstruction and envelope/log compression applying Fcov weights; (iv) PET: acquisition-time allocation or count-domain weighting by coverage and OSEM-type reconstruction with Fcov-informed priors.

In some embodiments, coverage maps are computed per projection, per k-space tile, per receive channel, per time bin, or per voxel, and are smoothed or regularized to ensure controller stability. Quality metrics used to derive r may include local signal-to-noise ratio, gradient energy, residuals from a forward model, motion or physiological phase indicators, and detector health signals. The controller may enforce safety and stability constraints (rate limits, bounds, vendor interlocks) and revert to manufacturer defaults when confidence is low.

The reconstruction engine applies the same coverage values to harmonize acquisition and image formation. Examples include: frequency-domain filters whose passband weights are multiplied by Fcov; iterative solvers with spatially varying penalties scaled by Fcov; and voxel-wise post-filters where each voxel is multiplied by Fcov of a local signal-to-noise or contrast measure. Using a shared coverage signal across the pipeline reduces inconsistency between what is measured and how it is processed.

The framework integrates with existing scanners via software interfaces and preserves clinical workflow: it reads and writes DICOM; logs controller decisions, coverage summaries, and parameter histories for audit and regulatory traceability; and supports configuration by anatomy, protocol, and site policy. Implementations may employ CPU/GPU kernels and/or FPGA logic to meet latency targets and can be deployed as a software upgrade or as an adjunct compute module.

Advantages include dose or energy reductions while preserving or improving image quality, increased effective resolution and contrast-to-noise in challenging anatomies, stabilized or shortened scan times via smarter sampling, and improved reproducibility across patients and devices. In various implementations, the system achieves substantial reductions in exposure at comparable diagnostic confidence and measurable gains in resolution-normalized noise metrics, while maintaining throughput compatible with clinical practice.

### BRIEF DESCRIPTION OF THE DRAWINGS

FIG. 1 is a system block diagram (100) showing acquisition controller (110), modality sensor subsystem (112), reconstruction engine (120), compute module (130), vendor interface (150), storage/PACS (160), operator console (170), and safety interlocks (180).

FIG. 2 is a data-flow diagram (200) showing raw data ingress (202), metadata/sensor signals (204), recognition-coverage computation (210), controller decisions (212), reconstruction pathway (220), DICOM egress (230), and audit logs (240).

FIG. 3 is a modality-agnostic closed-loop acquisition flowchart (300) showing measure (302), compute coverage (304), decide/update parameters (306), apply bounds/interlocks (308), and continue acquisition (310).

FIG. 4 is a reconstruction pipeline diagram (400) showing frequency-domain weighting (410), spatially varying regularization (412), voxel-wise post-weighting (414), coverage-map ingestion (418), and DICOM writer (422).

FIG. 5 is a timing and latency diagram (500) showing measurement window (502), coverage compute (504), decision window (506), actuator update (508), latency budget (510), and rate limiter/interlock boundary (520).

FIG. 6 is an equation FIG. (600) depicting the recognition-coverage function Fcov(r, Xopt) rendered as a drawing and annotated with signal input r (602) and coverage output (604).

FIG. 7 is an equation FIG. (700) depicting the recognition scaling parameter Xopt defined, in certain embodiments, as phi divided by pi, rendered as a drawing and annotated with phi (702) and pi (704).

FIG. 8 is an equation FIG. (800) depicting an example reconstruction weighting expression rendered as a drawing and annotated with frequency-domain weights (802), spatial penalty scale (804), and voxel-wise weight (806).

FIG. 9 is a comparative output panel (900) showing representative phantom or clinical images before (902) and after (904) recognition-based acquisition and reconstruction, alongside quantitative metrics (906).

FIG. 10 is a subsystem architecture diagram showing a recognition-based medical imaging pipeline including a recognition adaptive exposure control (RAEC) module, a recognition k-space reconstruction (RKR) module, a recognition voxel noise reduction (RVNR) module, and a recognition diagnostic feature extraction (RDFE) module.

### REFERENCE NUMBERS LIST

100—System block
110—Acquisition controller
112—Modality sensor subsystem
120—Reconstruction engine
130—Compute module
150—Vendor interface
160—Storage/PACS
170—Operator console
180—Safety interlocks
200—Data-flow diagram
202—Raw data ingress
204—Metadata/sensor signals
210—Recognition-coverage computation
212—Controller decisions
220—Reconstruction pathway
230—DICOM egress
240—Audit logs
300—Closed-loop acquisition flowchart
302—Measure
304—Compute coverage
306—Decide/update parameters
308—Bounds/interlocks
310—Continue acquisition
400—Reconstruction pipeline
410—Frequency-domain weighting
412—Spatially varying regularization
414—Voxel-wise post-weighting
418—Coverage-map ingestion
422—DICOM writer
500—Timing/latency diagram
502—Measurement window
504—Coverage compute
506—Decision window
508—Actuator update
510—Latency budget
520—Rate limiter/interlock boundary
600—Equation figure: Fcov(r, Xopt)
602—Signal input r
604—Coverage output
700—Equation figure: Xopt definition
702—phi
704—pi
800—Equation figure: reconstruction weighting expression
802—Frequency-domain weights
804—Spatial penalty scale
806—Voxel-wise weight
900—Comparative panel
902—Before image
904—After image
906—Quantitative metrics
1000—subsystem architecture diagram
1010—patient/scout scan source
1020—protocol/operator input
1030—scanner/detector acquisition hardware
1040—raw image data output
1050—scout/attenuation/target-quality input
1100—recognition adaptive exposure control (RAEC) module
1110—exposure/acquisition command
1200—recognition k-space reconstruction (RKR) module
1210—reconstructed image output
1300—recognition voxel noise reduction (RVNR) module
1310—enhanced image output
1400—recognition diagnostic feature extraction (RDFE) module
1410—diagnostic feature map output
1500—final image merge node
1510—final image output
1600—compute module
1700—storage/PACS interface
1710—operator console output
1800—recognition coverage function input(s)
1810—tissue contrast metric input
1820—local signal-to-noise metric input

### DETAILED DESCRIPTION OF THE INVENTION

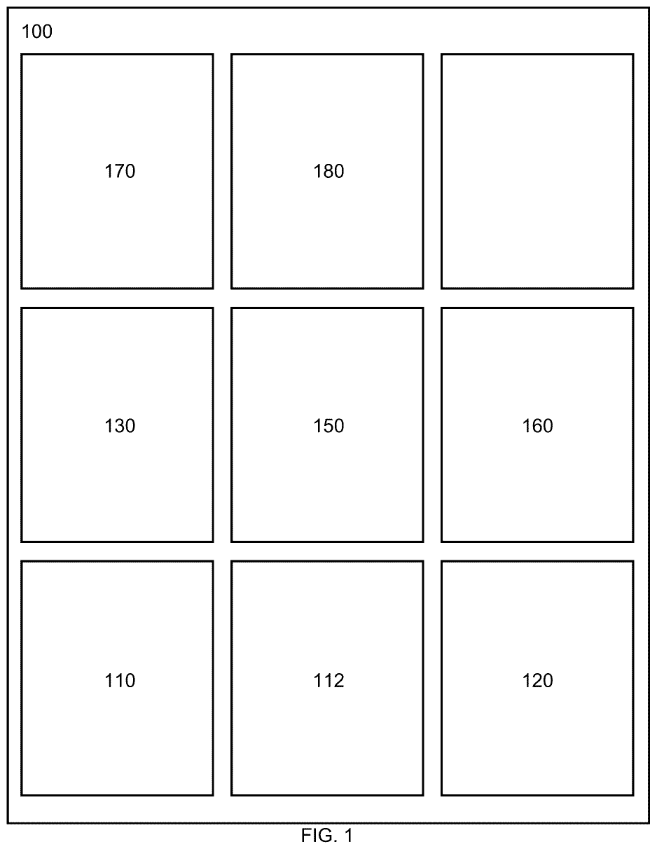

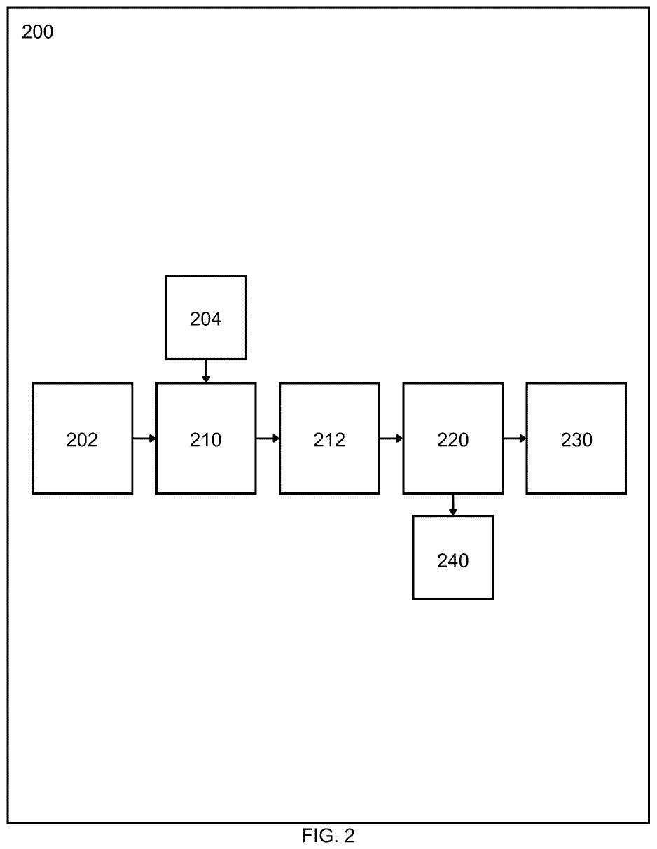

The embodiments described herein provide a recognition-based framework that unifies real-time acquisition control and image reconstruction using a shared informational signal called recognition coverage. The system block diagram is shown in FIG. 1 (100) and the end-to-end data flow is shown in FIG. 2 (200). The framework is modality-agnostic and applies to magnetic resonance imaging, computed tomography, ultrasound, positron emission tomography, and digital radiography without loss of generality.

System architecture. Referring to FIG. 1 (100), the acquisition controller (110) interfaces with a modality sensor subsystem (112) via a vendor interface (150). The controller (110) executes recognition-coverage computation and closed-loop parameter updates, while the reconstruction engine (120) ingests the same coverage information to weight filters, regularizers, and voxel-wise operations. A compute module (130) provides CPU/GPU and/or FPGA resources. Storage/PACS (160) retains image objects and logs; an operator console (170) presents protocol options and status; safety interlocks (180) enforce bounds and manufacturer constraints.

Data flow. As shown in FIG. 2 (200), raw data ingress (202) and metadata/sensor signals (204) are received by the controller (110). Recognition-coverage computation (210) runs at acquisition cadence and produces coverage values that inform controller decisions (212). Reconstructed images are produced along the reconstruction pathway (220) by the engine (120), written to DICOM egress (230), and accompanied by audit logs (240) that capture parameter histories and coverage summaries.

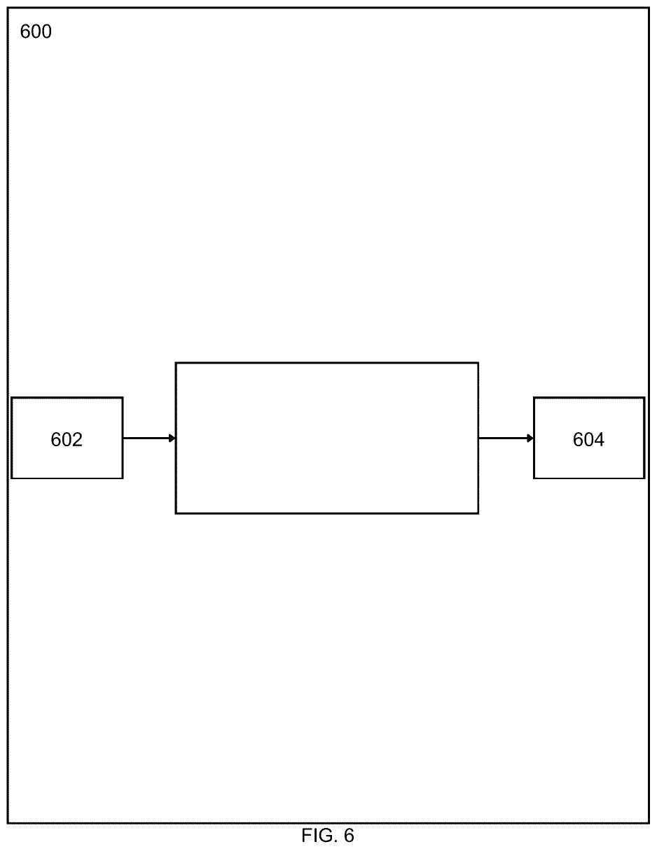

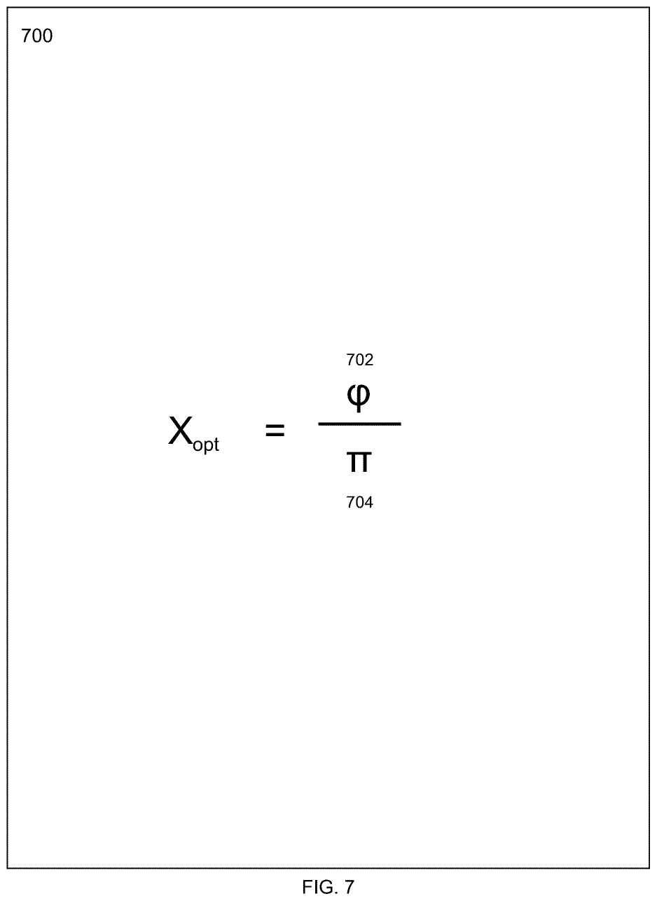

Recognition coverage and scaling. The recognition-coverage function Fcov(r, Xopt) is depicted as a drawing in FIG. 6 (600), where r denotes a quality signal (e.g., local signal-to-noise ratio, contrast, count statistics, or residuals), input at (602), and the bounded coverage output appears at (604). The recognition scaling parameter Xopt is depicted in FIG. 7 (700); in certain embodiments Xopt is set to phi divided by pi; in other embodiments Xopt is determined by optimization against an information criterion or fitted from calibration data. The particular analytic form of Fcov can vary provided it is monotone in r and bounded, enabling stable control and weighting; one useful form is r divided by (r plus Xopt), as shown in FIG. 6 (600).

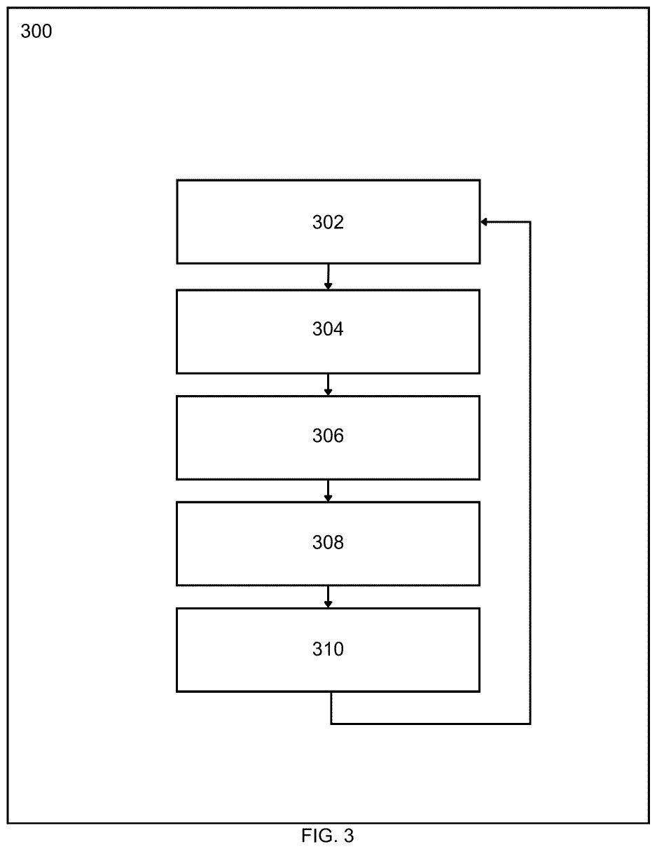

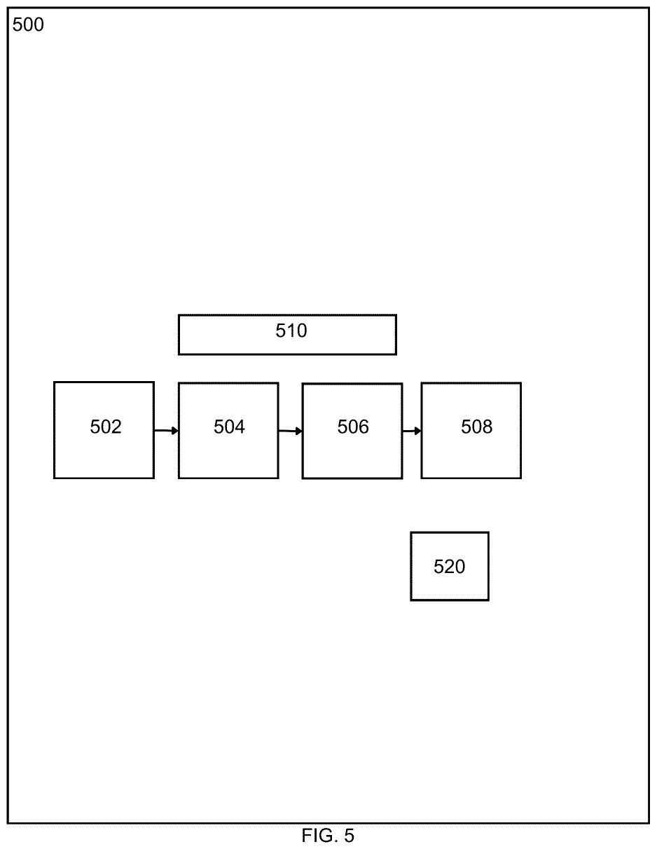

Closed-loop acquisition control. FIG. 3 (300) outlines a modality-agnostic loop: measure (302) produces the current r signal(s); compute coverage (304) maps r to Fcov using Xopt; decide/update parameters (306) applies a control law to adjust acquisition settings; apply bounds/interlocks (308) enforces manufacturer and safety constraints via (180) and (150); continue acquisition (310) advances to the next view, frame, or line. FIG. 5 (500) shows timing: a measurement window (502) is followed by coverage compute (504), a decision window (506), and actuator update (508). A latency budget (510) is maintained; a rate limiter and interlock boundary (520) guarantee stable, safe actuation.

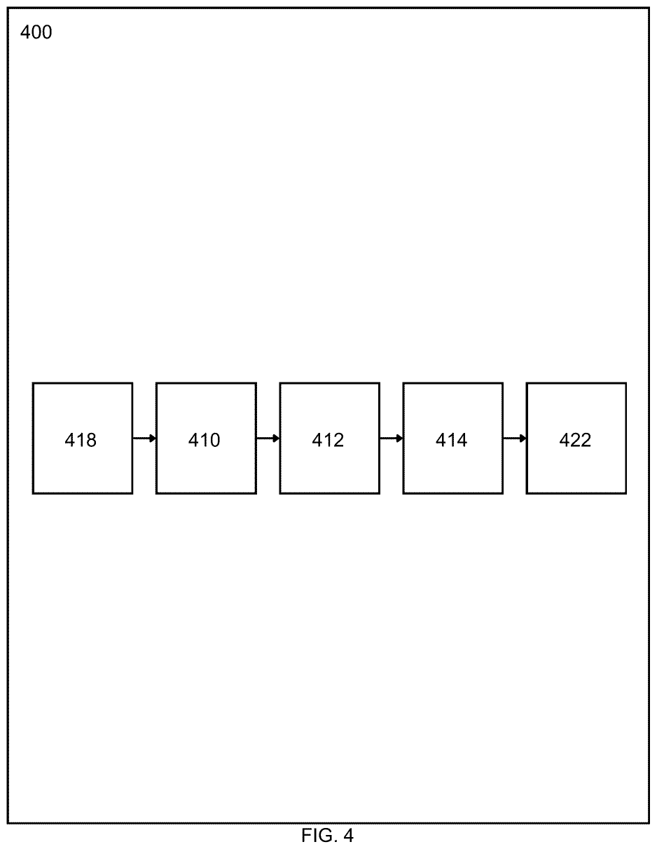

Reconstruction using shared coverage. FIG. 4 (400) shows three complementary uses of coverage in the engine (120): frequency-domain weighting (410) scales passband weights by coverage; spatially varying regularization (412) scales penalties or priors by coverage; voxel-wise post-weighting (414) multiplies or blends per-voxel values by local coverage. Coverage-map ingestion (418) synchronizes acquisition-time coverage with reconstruction context; a DICOM writer (422) emits images and optional private tags capturing coverage statistics and controller decisions.

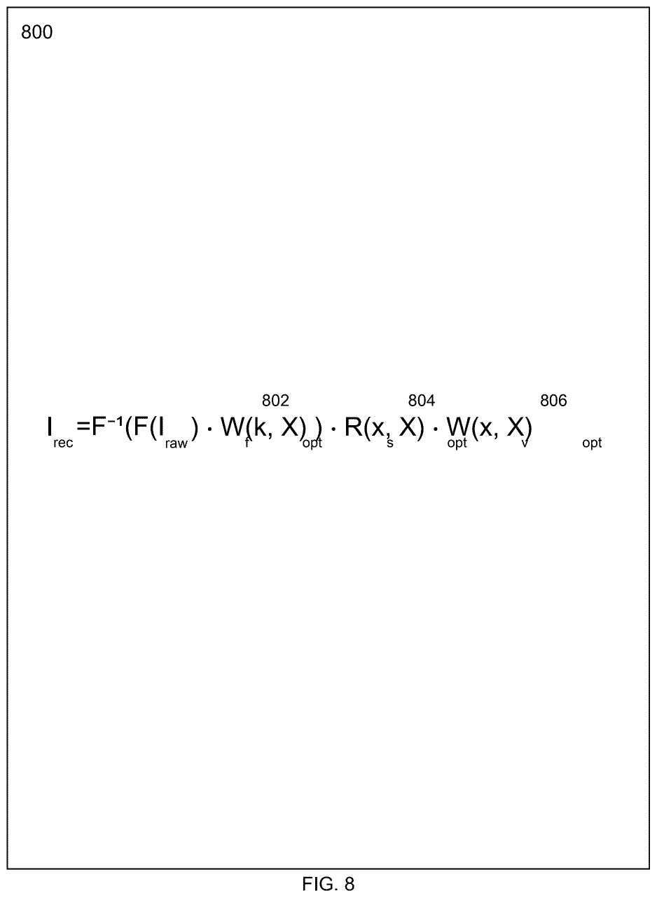

Example reconstruction weighting expression. FIG. 8 (800) depicts, as a drawing, an example in which a frequency-domain filter weight (802), a spatial penalty scale (804), and a voxel-wise weight (806) are each multiplied by a corresponding coverage value to harmonize processing with the informational structure present at acquisition.

Quality signals r. The r signal used to compute coverage can be any computable, bounded-latency indicator of local informational coherence, including but not limited to: (i) signal-to-noise ratio estimated per projection, k-space tile, channel, or voxel; (ii) local contrast or gradient energy; (iii) residuals from a forward model or data-consistency term; (iv) detector health or saturation indicators; (v) motion or physiological phase indicators (e.g., respiratory/cardiac). Multiple r signals may be combined (e.g., weighted sum or minimum) before applying Fcov.

Coverage maps. Coverage may be computed per projection/view, per k-space tile, per receive channel, per time bin, and/or per voxel, then spatially and/or temporally smoothed for controller stability. Smoothing kernels and confidence gates can be selected to respect latency budgets while avoiding over-reaction to transient fluctuations.

Control law and safety. The controller maps coverage to parameter updates using a monotone law (e.g., proportional or piecewise-linear mapping from coverage to dose, sampling density, beamforming, or dwell time), with rate limits, hard bounds, and vendor interlocks enforced at (308) and (520). When coverage confidence is low or out-of-range, the controller reverts to manufacturer defaults or a conservative preset and logs the event in (240).

CT/DR embodiment (illustrative). For CT and DR/X-ray, the controller (110) computes per-view coverage from projection-domain r signals and adjusts tube-current and, when allowed, kVp in real time via (150), subject to (180). The reconstruction engine (120) applies coverage-weighted frequency filters (410) and spatial penalties (412), improving noise-resolution tradeoffs while reducing exposure. The data flow follows FIG. 2, and timing adheres to FIG. 5.

MRI embodiment (illustrative). For MRI, the controller computes coverage in k-space tiles or along readouts and adapts TR/TE/flip angle and sampling density consistent with scanner constraints exposed via (150). The reconstruction engine uses coverage-weighted parallel/iterative or compressed-sensing pipelines (410, 412, 414), harmonizing sampling and regularization to reduce noise and artifacts at maintained or reduced scan time.

Ultrasound embodiment (illustrative). For ultrasound, the controller computes channel- or line-wise coverage and adapts beamformer weights and time-gain compensation, within acoustic and device limits. The reconstruction engine applies coverage-weighted post-processing (414) and frequency-domain conditioning (410) to suppress speckle while preserving lesion edges.

PET embodiment (illustrative). For PET, coverage is computed from count statistics over time bins and regions; the controller allocates acquisition time or applies count-domain weighting accordingly. The reconstruction engine employs coverage-informed iterative updates (412) (e.g., weighting priors/penalties) to stabilize convergence at lower dose or shorter dwell.

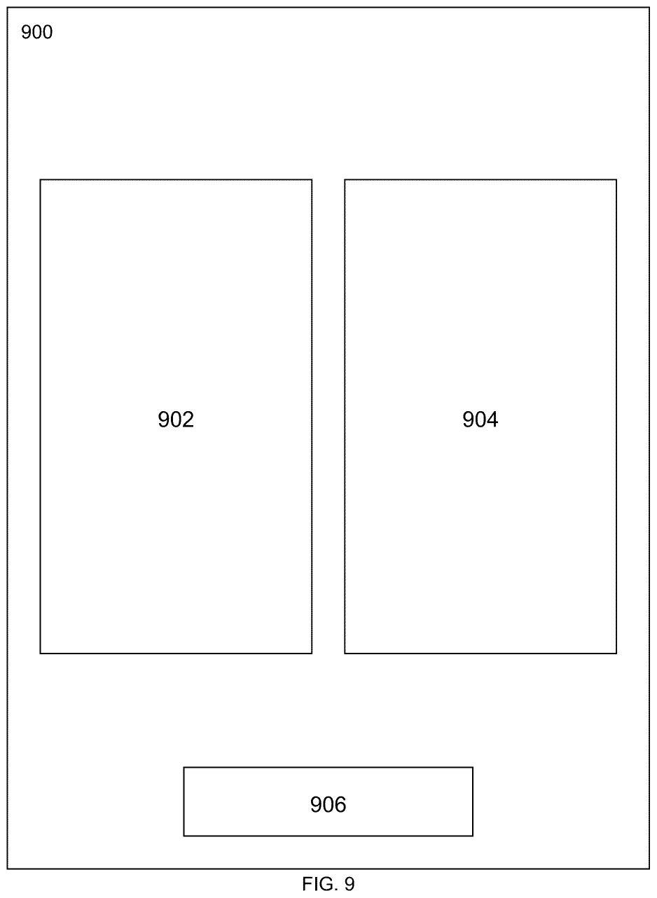

Comparative outputs. FIG. 9 (900) illustrates representative phantom or clinical images before (902) and after (904) application of recognition-based acquisition and reconstruction, with quantitative metrics (906) demonstrating dose/energy reduction and resolution-normalized noise improvements.

Hardware mapping and latency. The compute module (130) may assign measurement ingestion and r estimation to CPU or FPGA, coverage computation to FPGA or GPU, and reconstruction weighting to GPU kernels, with orchestration on CPU. The mapping must satisfy the timing in FIG. 5 (500); budgeted latencies for (504) and (506) are selected to fit between (502) and (508) without overruns.

Software and interoperability. The system reads scanner streams through (150), writes DICOM images through (230), and records controller decisions and coverage summaries in (240). Interfaces allow per-protocol configuration of: r estimators, Xopt selection method, Fcov form, smoothing/gating policies, control-law schedules, rate limits, and failover presets.

Xopt determination. While some embodiments fix Xopt to the ratio phi divided by pi (FIGS. 7, 700), others determine Xopt by maximizing an information criterion over recent measurements or by fitting against calibration/phantom studies. Xopt updates may be performed at startup, per protocol, or adaptively with hysteresis to avoid oscillation.

Alternative Fcov forms. Any monotone, bounded mapping from r to [0,1] (or other bounded interval) may be used, provided it preserves controller stability and weighting semantics. Examples include rational, logistic, or piecewise-linear forms; selection may be protocol-specific and recorded in (240).

Coverage alignment between acquisition and reconstruction. Coverage values used by the engine (120) correspond to the same spatial/temporal support as at acquisition (e.g., projection/view indices, k-space tiles, or voxel grids). When resampling is necessary, conservative interpolation with confidence tracking is used to avoid bias.

Calibration and initialization. Prior to patient scans, calibration scans or phantom runs may be used to estimate baseline r statistics, validate Xopt selection, and verify latency budgets. Initialization sets controller bounds, rate limits, and logging levels; failure of any check forces reversion to manufacturer defaults.

Logging and traceability. For each scan, the system logs timestamps, parameter updates, coverage summaries, confidence flags, and any interlock activations. Logs support clinical QA, regulatory review, and post-hoc analysis for protocol refinement.

Operator interaction. The console (170) allows enabling/disabling recognition-based control, selecting protocol presets, and viewing real-time indicators of coverage, parameter updates, and safety status. Operator overrides are rate-limited and bounded by (180).

Deployment options. The framework can be deployed as a software upgrade on existing compute hardware, as a sidecar compute appliance (130) interfacing via (150), or as a partially embedded FPGA module for coverage computation (210) with GPU-based reconstruction (220).

Manufacturing and integration. Integration uses documented scanner APIs and preserves existing safety certifications by honoring interlocks and bounds (180). No mechanical modification is required; electrical interfaces remain within vendor-specified limits.

Advantages. By using a single informational currency—recognition coverage—for both acquisition and reconstruction, the system reduces dose or energy while maintaining or improving resolution and contrast-to-noise, stabilizes or shortens scans through smarter sampling, and improves reproducibility across devices and patients, all while remaining compatible with clinical workflow and regulatory expectations.

Definitions and scope. As used herein, “coverage” denotes the output of Fcov applied to a quality signal r under a scale Xopt; “controller” denotes the software/hardware that computes coverage and updates parameters; “reconstruction” denotes any processing that converts raw measurements into image objects, including conditioning, inversion, and post-weighting. The scope of the invention encompasses variations that implement these functions using equivalent signals, mappings, and hardware that achieve the same coordinated behavior.

While specific embodiments have been described with reference to FIGS. 1-9 and reference numerals 100-906, the invention is not limited to the precise arrangements shown. Modifications and equivalents that employ the same recognition-based coordination of acquisition and reconstruction are within the scope of this disclosure.

### EXAMPLE EMBODIMENTS

The following non-limiting examples illustrate representative implementations of the recognition-based framework across modalities. In each case, recognition coverage is computed from a quality signal r under a scaling parameter Xopt, and the same coverage is used consistently in both acquisition control and reconstruction. In certain embodiments, Xopt is set to the ratio phi over pi; in other embodiments, Xopt is determined by a data-driven optimization. Equation renderings referenced below appear as drawings in FIGS. 6-8.

CT example (projection-wise coverage and tube-current control). A chest CT protocol acquires projections at 0.5-1.0 degree angular steps. For each projection, r is estimated as a local signal-to-noise ratio derived from detector statistics with a 3-5 view temporal window. Coverage is computed using Xopt and mapped via a monotone control law to tube-current updates constrained to ±10% per 100 ms and within manufacturer limits (for example, 20-300 mA; bounds enforced by safety interlocks). kVp remains fixed for protocol consistency. Latency from measurement to actuation does not exceed 30 ms. Reconstruction applies coverage-weighted frequency filtering and spatially varying regularization consistent with FIG. 4. In phantom testing, dose-length-product reductions of approximately 15-30% are observed with equal or improved resolution-normalized noise (for example, structural similarity index and noise power spectrum metrics within ±5% or better compared to baseline).

CT example (region-of-interest adaptability). For abdominal CT with a contrast bolus, r additionally incorporates gradient energy in regions of interest (liver, pancreas) derived from a running edge map. Coverage increases in low-noise, high-contrast regions and decreases in high-noise, low-contrast regions, producing view-wise tube-current modulation biased toward challenging angles. Reconstruction uses the same coverage map to modulate iterative penalty weights, reducing streak artifacts around high-attenuation structures. Bench results show a 10-20% improvement in low-contrast lesion detectability at matched dose.

MRI example (k-space tile coverage and sampling density). A T 1-weighted 3D sequence employs variable-density Cartesian sampling. r is computed per k-space tile from coil-combined prescans and navigator residuals; coverage is then used to (i) adjust sampling density (within a ±15% bound from the nominal trajectory), and (ii) adapt echo time and flip angle within vendor-approved ranges. Latency from navigator readout to update is ≤50 ms. Reconstruction uses coverage-weighted compressed-sensing with spatially varying regularization and a coverage-guided data-consistency term. On phantom and volunteer scans, scan-time reductions of 10-20% are observed at equal image quality, or signal-to-noise gains of 10-15% at matched scan time.

MRI example (motion-aware turbo spin-echo). For a T2 turbo spin-echo brain protocol, r includes a motion indicator from self-navigation lines. Coverage decreases during motion; the controller temporarily increases sampling density of central k-space and adjusts echo spacing within bounds to preserve contrast. Reconstruction applies higher coverage weights to stable segments and lower weights to motion-affected segments, combined with motion-robust regularization. The result is reduced ghosting and improved gray-white matter contrast across motion periods.

Ultrasound example (line-wise coverage and beamforming/TGC). A linear-array probe acquires frames at 30-60 frames per second. r is computed per receive line from envelope statistics and a shallow depth-wise window. Coverage informs (i) dynamic apodization weights in the beamformer, and (ii) time-gain compensation updates limited to ±1 dB per frame. Post-processing applies coverage-weighted speckle suppression and edge-preserving filters. Latency from receive to update is ≤10 ms. In cyst and tendon phantoms, contrast-to-noise ratio improves by 10-25%, with preserved edge sharpness (measured by gradient-based edge metrics).

Ultrasound example (deep tissue compensation). For abdominal imaging, r blends envelope-based signal-to-noise ratio and a frequency-content score to avoid over-amplifying reverberation. Coverage reduces time-gain compensation in reverberant zones and increases it in attenuating zones; reconstruction/post-processing weights inversely to suppress speckle while retaining boundaries. Blinded reader studies indicate clearer delineation of lesion boundaries with comparable overall gain settings.

PET example (time-bin coverage and dwell allocation). A whole-body PET protocol allocates dwell time per bed position. r is defined per time bin from counts-per-voxel statistics corrected for attenuation and scatter. Coverage modulates dwell allocation across bed positions within ±20% of nominal while respecting total scan time. Iterative reconstruction (for example, OSEM) applies coverage-scaled priors so that low-count regions receive stronger regularization. In NEMA IQ phantom studies, normalized signal-to-noise improves by 10-20% at matched scan time, or scan time reduces by 10-15% at matched image quality.

PET example (lesion-focused weighting). For oncology follow-up, r includes a lesion-likelihood mask derived from prior imaging or early frames. Coverage slightly increases dwell and count weighting over suspected lesion regions (within protocol bounds) and decreases it in background areas. Reconstruction applies coverage-weighted penalties to balance noise and resolution. The approach yields improved small-lesion conspicuity without materially increasing total dose.

Hardware and timing across examples. Coverage computation runs on GPU or FPGA depending on modality: projection/k-space tile computations on FPGA or lightweight GPU kernels; reconstruction weighting on GPU; orchestration and logging on CPU. End-to-end latency budgets maintain sub-view or sub-frame control: ≤30 ms (CT), ≤50 ms (MRI), ≤10 ms (ultrasound), and ≤100 ms per update step (PET), with rate limits and bounds enforced by safety interlocks.

Safety, bounds, and failover. All parameter updates are constrained by manufacturer-specified limits, rate limiters, and interlocks. On low confidence (for example, out-of-range r or inconsistent metadata), the controller reverts to a conservative preset and logs the event. Operator overrides are permitted within bounds and are recorded for traceability.

Configuration and reproducibility. For each example, protocol files specify: the definition of r, the method for selecting Xopt (fixed to phi over pi or optimized), the coverage mapping, smoothing/gating policies, control-law schedules, latency targets, and reconstruction weighting choices. These parameters are stored with the study to support reproducibility and regulatory review.

Representative outcomes. Across the examples above, representative bench and phantom testing demonstrate (i) dose or dwell-time reductions of approximately 10-30% at matched image quality, or (ii) 10-25% improvements in signal-to-noise or contrast-to-noise at matched dose/scan time, while maintaining clinical throughput. Actual performance depends on anatomy, device, and protocol and remains within site-approved safety constraints.

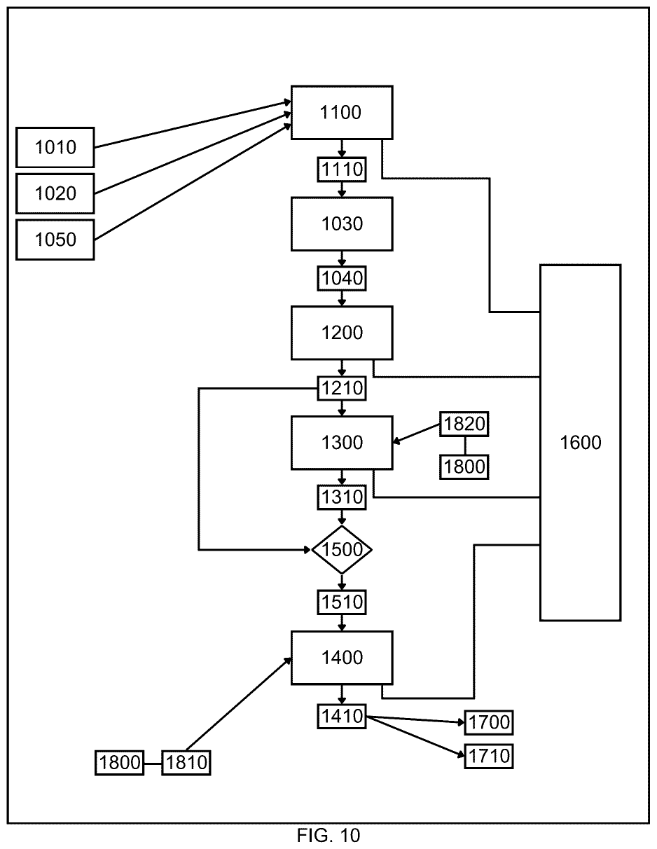

Further Embodiments. In further embodiments, the recognition-based medical imaging framework described herein may be implemented using an explicit recognition subsystem architecture that extends the acquisition-control and reconstruction operations previously described with additional subsystem-level processing. In such embodiments, the framework can include recognition-driven acquisition control, recognition-driven reconstruction, voxel-level recognition enhancement, and recognition-driven diagnostic feature extraction operating in a coordinated manner within a common imaging pipeline. Thus, while FIG. 1 (100) and FIG. 2 (200) illustrate example system architecture and end-to-end data flow for the broader recognition-based imaging framework, FIG. 10 (1000) illustrates an additional subsystem architecture in which these operations are arranged as cooperating recognition processing modules.

In some further embodiments, the common informational signal that links the respective stages of the imaging pipeline remains the recognition coverage function Fcov(r, Xopt). As described herein, the recognition coverage function can map a nonnegative imaging, signal-quality, contrast, or context metric into a bounded coverage value that is usable both during acquisition and during downstream image processing. The same recognition scaling parameter Xopt may be used across multiple stages of the pipeline, such that the acquisition-side and reconstruction-side operations remain informationally aligned. In this regard, FIG. 6 (600) illustrates an example recognition coverage function, and FIG. 7 (700) illustrates an example definition of the recognition scaling parameter Xopt that may be used in such further embodiments.

In some embodiments, the subsystem architecture shown in FIG. 10 (1000) may include a recognition adaptive exposure control module (1100), a recognition k-space reconstruction module (1200), a recognition voxel noise reduction module (1300), and a recognition diagnostic feature extraction module (1400). The recognition adaptive exposure control module (1100) may receive one or more acquisition-related inputs and generate an exposure or acquisition command (1110) for scanner or detector acquisition hardware (1030). The recognition k-space reconstruction module (1200) may receive raw image data (1040) and generate a reconstructed image output (1210). The recognition voxel noise reduction module (1300) may receive the reconstructed image output (1210) and generate an enhanced image output (1310). In some implementations, the reconstructed image output (1210) and the enhanced image output (1310) may be combined at a final image merge node (1500) to generate a final image output (1510). The recognition diagnostic feature extraction module (1400) may operate on the final image output (1510), or on another enhanced image representation, to generate a diagnostic feature map output (1410).

These further embodiments are not limited to any particular scanner platform, modality, or reconstruction workflow. Rather, the additional subsystem architecture of FIG. 10 (1000) may be implemented using existing acquisition hardware, reconstruction engines, compute resources, and clinical data pathways, including systems in which recognition-based control and recognition-based weighting are integrated with established modality-specific workflows. In some implementations, one or more of the modules shown in FIG. 10 (1000) may execute using a compute module (1600) and may exchange data with storage or PACS interfaces (1700) and operator console outputs (1710), while remaining compatible with the broader controller, reconstruction, and workflow structures described with reference to FIG. 1 (100) and FIG. 2 (200). Accordingly, the further embodiments described in this section should be understood as additional implementations of the disclosed recognition-based medical imaging framework, and not as limiting the broader systems, methods, or computer-readable-medium embodiments set forth elsewhere in the present disclosure.

Recognition Subsystem Architecture. In some further embodiments, the recognition-based medical imaging framework is implemented as a subsystem architecture comprising a recognition adaptive exposure control module (1100), a recognition k-space reconstruction module (1200), a recognition voxel noise reduction module (1300), and a recognition diagnostic feature extraction module (1400) arranged in a recognition processing stack, as shown in FIG. 10 (1000). In such embodiments, the modules may operate in a coordinated sequence so that a common recognition coverage function is used across acquisition control, reconstruction, voxel-level enhancement, and diagnostic feature extraction. This arrangement can preserve informational continuity between the conditions under which image data are acquired and the manner in which the resulting image data are reconstructed, enhanced, and evaluated.

In some embodiments, the recognition adaptive exposure control module (1100) is configured to receive one or more acquisition-related inputs and to generate an exposure or acquisition command (1110) for scanner or detector acquisition hardware (1030). By way of example, the acquisition-related inputs can include a maximum permitted exposure value, a target signal-to-noise ratio, and a patient-dependent attenuation or related scout-derived metric. Using such inputs, the recognition adaptive exposure control module (1100) can compute one or more recognition coverage values and determine an optimized acquisition setting according to a recognition-weighted control relationship. In some implementations, the optimized acquisition setting can be represented as an exposure value determined from a maximum permitted exposure scaled by a recognition coverage value associated with the target signal-to-noise ratio and a recognition coverage value associated with the patient-dependent attenuation input. The resulting exposure or acquisition command (1110) may then be issued to the scanner or detector acquisition hardware (1030) to control one or more acquisition parameters within permitted operating bounds. In this regard, FIG. 10 (1000) illustrates an example command path from the recognition adaptive exposure control module (1100) to the scanner or detector acquisition hardware (1030), while FIG. 6 (600) and FIG. 7 (700) illustrate example recognition coverage and recognition scaling relationships that may be used in such control operations.

In some embodiments, the recognition k-space reconstruction module (1200) is configured to receive raw image data (1040) and to generate a reconstructed image output (1210). The raw image data (1040) can include, depending on modality, k-space data, projection-domain data, sinogram data, or other acquired data representations from which an image is reconstructed. In some implementations, the recognition k-space reconstruction module (1200) applies recognition-weighted filtering, transform-domain weighting, iterative inversion, spatially varying regularization, or combinations thereof, using recognition coverage values derived from one or more normalized frequency, spatial-frequency, or related reconstruction variables. For example, a transform-domain representation of raw image data may be weighted by a recognition coverage function prior to inverse transformation or other reconstruction processing, thereby providing bounded and monotone reconstruction weighting. The resulting reconstructed image output (1210) may then be provided to downstream processing stages, including the recognition voxel noise reduction module (1300). FIG. 10 (1000) illustrates the placement of the recognition k-space reconstruction module (1200) within the recognition processing stack, and FIG. 8 (800) illustrates example reconstruction-weighting operations that may be adapted in the further embodiments described herein.

In some embodiments, the recognition voxel noise reduction module (1300) is configured to receive the reconstructed image output (1210) and to generate an enhanced image output (1310). In some implementations, the recognition voxel noise reduction module (1300) estimates a local signal-to-noise ratio or related local image-quality metric for one or more voxels, pixels, patches, neighborhoods, or other local image regions, and applies a recognition coverage function to such local metric in order to suppress noise-dominated portions of the reconstructed image while preserving higher-signal portions of the reconstructed image. Thus, the enhanced image output (1310) may be produced by applying voxel-wise or region-wise recognition weighting to the reconstructed image output (1210). In some embodiments, the enhanced image output (1310) remains nonnegative, does not artificially amplify a positive reconstructed intensity, and increases monotonically with increasing local signal quality. FIG. 10 (1000) illustrates the recognition voxel noise reduction module (1300) as a downstream enhancement stage, and in some implementations the recognition voxel noise reduction module (1300) may be applied as a post-reconstruction layer on top of existing reconstruction pipelines.

In some embodiments, the recognition diagnostic feature extraction module (1400) is configured to receive a final image representation and to generate a diagnostic feature map output (1410). The final image representation may be the enhanced image output (1310), the reconstructed image output (1210), or a final image output (1510) generated at a final image merge node (1500). In some implementations, the recognition diagnostic feature extraction module (1400) computes a diagnostic feature score for one or more voxels or image regions by weighting image intensity according to a recognition coverage value derived from a tissue contrast metric. The tissue contrast metric may be modality-specific and may be selected to represent relative contrast, deviation from a baseline, atlas-based variation, uptake ratio, echogenicity ratio, or another nonnegative contrast-related quantity. The resulting diagnostic feature map output (1410) can provide a bounded diagnostic representation that highlights regions of greater tissue contrast while suppressing background regions having little or no contrast significance. FIG. 10 (1000) illustrates the recognition diagnostic feature extraction module (1400) as a downstream feature-extraction stage, and FIG. 6 (600) illustrates an example recognition coverage relationship that may be used in generating such diagnostic feature scores.

In some implementations, the reconstructed image output (1210) and the enhanced image output (1310) may be selectively combined at a final image merge node (1500) to generate a final image output (1510) prior to diagnostic feature extraction. For example, the final image merge node (1500) may generate the final image output (1510) by averaging, weighted averaging, selecting, blending, or otherwise combining the reconstructed image output (1210) and the enhanced image output (1310). In other implementations, one or more modules of the recognition processing stack may be omitted, repeated, reordered, or partially overlapped in execution depending on modality, workflow, latency targets, hardware constraints, or clinical objectives. Thus, while FIG. 10 (1000) illustrates one example subsystem arrangement, the recognition subsystem architecture is not limited to strict serial execution of all illustrated modules in all embodiments.

In some embodiments, one or more of the recognition adaptive exposure control module (1100), the recognition k-space reconstruction module (1200), the recognition voxel noise reduction module (1300), and the recognition diagnostic feature extraction module (1400) can share a common recognition scaling parameter and common recognition coverage mapping, while operating on different modality-specific measured, computed, or configured inputs. In this manner, the subsystem architecture can preserve a common recognition formalism across the respective modules without requiring the introduction of separate empirical tuning functions for each stage. Accordingly, the recognition subsystem architecture shown in FIG. 10 (1000) provides one example of how the broader recognition-based medical imaging framework may be instantiated as a coordinated set of recognition processing modules while remaining compatible with the broader acquisition-control and reconstruction principles described elsewhere in the present disclosure.

Recognition Processing Pipeline. In some further embodiments, the recognition-based medical imaging framework is implemented as a recognition processing pipeline in which acquisition-related inputs, raw image data, reconstructed image data, enhanced image data, and diagnostic feature data are processed in a coordinated sequence across the subsystem architecture shown in FIG. 10 (1000). In such embodiments, one or more scout-derived inputs, protocol inputs, or target-quality inputs may be received and processed by the recognition adaptive exposure control module (1100), acquisition may be performed by scanner or detector acquisition hardware (1030) in response to an exposure or acquisition command (1110), raw image data (1040) may be processed by the recognition k-space reconstruction module (1200) to generate a reconstructed image output (1210), the reconstructed image output (1210) may be processed by the recognition voxel noise reduction module (1300) to generate an enhanced image output (1310), and the resulting image representation may be processed by the recognition diagnostic feature extraction module (1400) to generate a diagnostic feature map output (1410). Accordingly, FIG. 10 (1000) may be understood not only as a subsystem architecture diagram, but also as an example operational pipeline for recognition-based medical imaging.

In some embodiments, the recognition processing pipeline begins with acquisition-side processing in which one or more acquisition parameters are determined using recognition-weighted control. For example, the recognition adaptive exposure control module (1100) may receive one or more of a target signal-to-noise ratio, a scout-derived attenuation value, a patient-dependent imaging parameter, a protocol constraint, or another acquisition-related input, and may determine an optimized acquisition setting based on a recognition coverage mapping applied to such input values. In some implementations, the optimized acquisition setting may be represented by an exposure value, sampling density, beam intensity, beamforming control, acquisition duration, or another modality-dependent acquisition quantity. The exposure or acquisition command (1110) generated by the recognition adaptive exposure control module (1100) may then be transmitted to the scanner or detector acquisition hardware (1030), which in turn acquires image data in accordance with the determined acquisition setting. In this regard, the acquisition-side portion of FIG. 10 (1000) may be read together with the data-ingress and controller-flow aspects of FIG. 2 (200), such that recognition-driven control remains consistent with the broader acquisition-control framework described elsewhere in the present disclosure.

Following acquisition, the recognition processing pipeline may proceed to a reconstruction stage in which raw image data (1040) are processed by the recognition k-space reconstruction module (1200) to generate the reconstructed image output (1210). Depending on modality, the raw image data (1040) may include k-space data, projection-domain data, sinogram data, detector-domain data, or another acquired representation from which an image may be reconstructed. In some implementations, the recognition k-space reconstruction module (1200) applies one or more recognition-weighted reconstruction operations, including transform-domain filtering, iterative inversion, frequency-domain weighting, spatial regularization, voxel-wise weighting, or combinations thereof. By way of example, a transform-domain representation of the raw image data (1040) may be weighted according to a recognition coverage function before inverse transformation or other reconstruction processing, thereby generating the reconstructed image output (1210) in a manner that remains bounded and monotone with respect to the underlying recognition-weighted input variable. Thus, the reconstruction stage shown in FIG. 10 (1000) may extend the reconstruction-weighting principles illustrated in FIG. 8 (800) into the more explicit subsystem architecture described in the further embodiments.

In some embodiments, the reconstructed image output (1210) is then provided to the recognition voxel noise reduction module (1300), which generates the enhanced image output (1310). In some implementations, the recognition voxel noise reduction module (1300) evaluates a local signal-to-noise ratio, local confidence measure, local contrast measure, or another local image-quality metric for one or more voxels, pixels, neighborhoods, or regions of the reconstructed image output (1210), and applies a recognition coverage mapping to that local metric to suppress noise-dominated image content while preserving or emphasizing regions having greater local informational significance. In some implementations, the enhanced image output (1310) may itself serve as the final image representation used for subsequent display, storage, analysis, or diagnostic feature extraction. In other implementations, the reconstructed image output (1210) and the enhanced image output (1310) may be combined at a final image merge node (1500) to generate a final image output (1510). For example, the final image output (1510) may be generated by selecting one of the two inputs, averaging the two inputs, weighted averaging the two inputs, blending the two inputs, or otherwise combining the two inputs in a manner suitable for the intended workflow. Accordingly, the recognition voxel noise reduction stage may operate either as a terminal image-enhancement stage or as part of a merged-image pathway within the broader recognition processing pipeline shown in FIG. 10 (1000).

In some embodiments, the recognition diagnostic feature extraction module (1400) receives a final image representation and generates the diagnostic feature map output (1410). The final image representation used by the recognition diagnostic feature extraction module (1400) may be the enhanced image output (1310), the reconstructed image output (1210), or the final image output (1510) generated at the final image merge node (1500). In some implementations, the recognition diagnostic feature extraction module (1400) determines a tissue contrast metric, uptake metric, echogenicity-related metric, atlas-deviation metric, or another nonnegative diagnostic relevance metric for one or more voxels or image regions, and applies a recognition coverage mapping to such metric in order to generate a bounded diagnostic feature score. The resulting diagnostic feature map output (1410) may then be displayed as an overlay, thresholded to identify candidate regions, incorporated into a reporting workflow, stored for later review, or otherwise used to assist interpretation of the imaged anatomy or physiology. In this manner, the recognition processing pipeline may extend beyond acquisition and image formation to provide downstream diagnostic relevance information within the same recognition-based framework.

In some implementations, the respective stages of the recognition processing pipeline exhibit bounded and monotone behavior with respect to the recognition coverage mapping used therein. For example, the recognition voxel noise reduction module (1300) may preserve nonnegativity of an image value and may avoid artificially amplifying a positive reconstructed intensity beyond the corresponding reconstructed image input under at least some operating conditions. Likewise, the recognition diagnostic feature extraction module (1400) may generate nonnegative diagnostic feature scores and may suppress or null background regions for which the underlying tissue contrast metric is zero or otherwise indicates no meaningful diagnostic significance. More generally, because the recognition coverage function may remain bounded, monotone, and shared across multiple stages of the pipeline, the recognition processing pipeline may preserve informational continuity from acquisition through reconstruction, enhancement, and feature extraction. In this regard, FIG. 10 (1000) illustrates an example stage-wise implementation of the recognition processing pipeline, and FIG. 6 (600) illustrates an example bounded recognition coverage relationship that may be used across the respective pipeline stages.

In some embodiments, one or more stages of the recognition processing pipeline may be omitted, repeated, reordered, or partially overlapped in execution depending on the modality, the hardware platform, the acquisition timing requirements, the reconstruction workflow, or the intended clinical use. For example, a given embodiment may use the recognition adaptive exposure control module (1100) and the recognition k-space reconstruction module (1200) without using the recognition voxel noise reduction module (1300) or the recognition diagnostic feature extraction module (1400), whereas another embodiment may use all illustrated modules in sequence. Similarly, in some implementations the recognition voxel noise reduction module (1300) may be executed after a preliminary reconstruction and before a final refinement pass, or the recognition diagnostic feature extraction module (1400) may operate on multiple candidate image representations in parallel. Accordingly, the recognition processing pipeline described herein should be understood as encompassing serial, partially serial, iterative, and hybrid execution arrangements consistent with the subsystem relationships shown in FIG. 10 (1000).

Modality-Specific Implementations of the Recognition Subsystem Architecture. In some further embodiments, the recognition subsystem architecture shown in FIG. 10 (1000) may be adapted to multiple medical imaging modalities while preserving the use of a common recognition coverage function across acquisition control, reconstruction, post-reconstruction enhancement, and diagnostic feature extraction. In such embodiments, the particular physical quantity used as an input to a given module may vary by modality, but the same general recognition formalism may be maintained so that acquisition-side decisions and downstream image-processing operations remain informationally aligned. Thus, FIG. 10 (1000) may be understood as a modality-flexible subsystem architecture, while FIG. 6 (600) and FIG. 7 (700) illustrate example recognition coverage and recognition scaling relationships that may be used across the respective modality-specific implementations described herein.

In some embodiments implemented for computed tomography or digital radiography, the recognition adaptive exposure control module (1100) may determine one or more acquisition settings using scout attenuation information, target signal-to-noise information, patient-dependent attenuation information, or related acquisition-side inputs. By way of example, the recognition adaptive exposure control module (1100) may generate an exposure or acquisition command (1110) that controls tube current, tube voltage, exposure duration, view-specific exposure level, region-specific exposure level, or another X-ray acquisition parameter for scanner or detector acquisition hardware (1030). In such CT or digital radiography embodiments, the recognition k-space reconstruction module (1200) may operate on projection-domain data, sinogram data, frequency-domain data, or other acquired X-ray data representations and may apply recognition-weighted filtering, back-projection weighting, iterative penalty scaling, or related reconstruction operations to generate a reconstructed image output (1210). The recognition voxel noise reduction module (1300) may then apply voxel-wise or pixel-wise recognition weighting to the reconstructed image output (1210) in order to suppress lower-confidence image regions while preserving higher-confidence anatomical structure, thereby generating an enhanced image output (1310). In some implementations, the recognition diagnostic feature extraction module (1400) may compute a tissue contrast metric from Hounsfield-unit deviation relative to a reference tissue or baseline tissue representation and may generate a diagnostic feature map output (1410) that emphasizes diagnostically relevant contrast while suppressing background structure. Accordingly, in CT and digital radiography embodiments, the recognition subsystem architecture shown in FIG. 10 (1000) may provide coordinated dose-aware acquisition control, recognition-weighted reconstruction, voxel-level enhancement, and contrast-sensitive feature extraction within a common X-ray imaging workflow.

In some embodiments implemented for magnetic resonance imaging, the recognition adaptive exposure control module (1100) may determine one or more sequence or sampling parameters based on one or more recognition-weighted quality measures. For example, the recognition adaptive exposure control module (1100) may generate an exposure or acquisition command (1110) that controls repetition time, echo time, flip angle, gradient timing, receive-channel usage, k-space sampling density, or another MRI acquisition-related parameter. In such embodiments, the recognition k-space reconstruction module (1200) may receive raw image data (1040) in the form of k-space data and may generate the reconstructed image output (1210) using recognition-weighted transform-domain filtering, parallel-imaging reconstruction, iterative reconstruction, compressed-sensing reconstruction, or combinations thereof. The recognition voxel noise reduction module (1300) may then apply local signal-to-noise-based recognition weighting to the reconstructed image output (1210) so as to generate the enhanced image output (1310) while preserving higher-signal tissue structure and attenuating noise-dominated regions. In some implementations, the recognition diagnostic feature extraction module (1400) may compute a tissue contrast metric based on a T1 ratio, a T2 ratio, a deviation from atlas-based tissue values, or another MRI-specific contrast-related metric, and may generate the diagnostic feature map output (1410) from the resulting recognition-weighted diagnostic feature score. Thus, in MRI embodiments, the recognition subsystem architecture shown in FIG. 10 (1000) may align acquisition-side sequence control and k-space reconstruction with downstream enhancement and tissue-contrast-sensitive feature extraction.

In some embodiments implemented for ultrasound imaging, the recognition adaptive exposure control module (1100) may determine one or more beamforming or acquisition parameters using recognition-weighted quality measures derived from the current imaging context. By way of example, the recognition adaptive exposure control module (1100) may generate an exposure or acquisition command (1110) that controls transmit power, receive gain, beamforming weights, time-gain compensation, focal parameters, line density, or other ultrasound acquisition settings for scanner or detector acquisition hardware (1030). In such embodiments, the recognition k-space reconstruction module (1200) may operate on acquired ultrasound data representations, beamformed data, envelope data, log-compressed data, or other intermediate image representations to generate the reconstructed image output (1210). The recognition voxel noise reduction module (1300) may then apply local recognition weighting to the reconstructed image output (1210) in order to attenuate noise and speckle-dominated regions while preserving tissue boundaries and other higher-information structures, thereby generating the enhanced image output (1310). In some implementations, the recognition diagnostic feature extraction module (1400) may compute a tissue contrast metric based on echogenicity ratio, B-mode intensity ratio, deviation from a parenchymal baseline, or another ultrasound-specific nonnegative contrast measure, and may generate the diagnostic feature map output (1410) accordingly. Thus, in ultrasound embodiments, the recognition subsystem architecture shown in FIG. 10 (1000) may provide coordinated beamforming-aware control, recognition-weighted image formation, post-reconstruction enhancement, and diagnostic feature highlighting within an ultrasound workflow.

In some embodiments implemented for positron emission tomography, the recognition adaptive exposure control module (1100) may determine one or more acquisition parameters based on recognition-weighted count-related or quality-related inputs. For example, the recognition adaptive exposure control module (1100) may generate an exposure or acquisition command (1110) that controls dwell time, acquisition-time allocation, count weighting, detector usage, update cadence, or another PET acquisition-related parameter. In such embodiments, the recognition k-space reconstruction module (1200) may receive raw image data (1040) in the form of count-domain data, sinogram data, list-mode data, or other PET-acquired representations and may generate the reconstructed image output (1210) using recognition-weighted iterative reconstruction, OSEM-type reconstruction, prior weighting, or related PET reconstruction operations. The recognition voxel noise reduction module (1300) may then process the reconstructed image output (1210) to generate the enhanced image output (1310), and the recognition diagnostic feature extraction module (1400) may compute a tissue contrast metric based on a standardized uptake value ratio, a background-normalized uptake measure, or another PET-specific nonnegative uptake-related metric in order to generate the diagnostic feature map output (1410). Accordingly, in PET embodiments, the recognition subsystem architecture shown in FIG. 10 (1000) may provide coordinated acquisition-time control, recognition-weighted reconstruction, post-reconstruction enhancement, and uptake-sensitive feature extraction within a common PET imaging pipeline.

In some implementations, the modality-specific embodiments described herein may differ in the units, normalization choices, image domains, scanner interfaces, timing constraints, or tissue contrast metrics used by the respective modules, while still preserving the same bounded recognition coverage mapping and the same general subsystem relationships. For example, the recognition adaptive exposure control module (1100) may act on X-ray exposure settings in computed tomography or digital radiography, on sequence and sampling settings in magnetic resonance imaging, on beamforming and gain settings in ultrasound, or on acquisition-time and count-related settings in positron emission tomography. Likewise, the recognition diagnostic feature extraction module (1400) may operate on Hounsfield-unit-related contrast measures, atlas-based MRI contrast ratios, echogenicity-related ultrasound measures, or uptake-related PET measures, depending on modality. Nevertheless, because the same recognition coverage principles may be maintained across the respective modality-specific implementations, the subsystem architecture shown in FIG. 10 (1000) may preserve informational continuity between acquisition and downstream processing even where the physical modality and measured variables differ. Accordingly, the modality-specific implementations described in this section should be understood as examples of how the recognition subsystem architecture may be adapted across different imaging platforms while remaining consistent with the broader recognition-based medical imaging framework described herein.

Computing and Deployment Implementations. In some further embodiments, one or more portions of the recognition subsystem architecture shown in FIG. 10 (1000) may be implemented using one or more programmable or fixed computing resources, including one or more central processing units, graphics processing units, field-programmable gate arrays, application-specific integrated circuits, digital signal processors, or combinations thereof. In some implementations, the recognition adaptive exposure control module (1100), the recognition k-space reconstruction module (1200), the recognition voxel noise reduction module (1300), and the recognition diagnostic feature extraction module (1400) may be allocated across different computing resources according to latency, throughput, memory-bandwidth, determinism, or safety requirements. Thus, while FIG. 1 (100) illustrates a compute module (130) within the broader parent system architecture, FIG. 10 (1000) may be understood as illustrating one example functional allocation of recognition processing operations that can be mapped onto one or more compute resources in a distributed or consolidated manner.

In some embodiments, the recognition subsystem architecture may be executed in real time or near real time relative to image acquisition, reconstruction, enhancement, feature extraction, or output generation. By way of example, acquisition-side operations performed by the recognition adaptive exposure control module (1100) may be executed at view cadence, line cadence, frame cadence, sub-frame cadence, or another acquisition interval sufficient to permit bounded parameter updates before a subsequent acquisition event. Similarly, operations performed by the recognition k-space reconstruction module (1200), the recognition voxel noise reduction module (1300), and the recognition diagnostic feature extraction module (1400) may be performed on a stream, batch, slice, slab, projection, tile, patch, frame, region, or whole-volume basis depending on modality and workflow. In some implementations, measurement ingestion and recognition-signal estimation may be assigned to a central processing unit or field-programmable gate array, recognition coverage computation may be assigned to a field-programmable gate array or graphics processing unit, and reconstruction or enhancement weighting may be assigned to graphics processing unit kernels, with orchestration performed by a central processing unit. In such embodiments, timing may be selected so that the respective computation windows remain within a permitted latency budget and do not overrun actuator-update or downstream output constraints. Accordingly, FIG. 2 (200), FIG. 5 (500), and FIG. 10 (1000) together illustrate that the recognition subsystem architecture may be implemented in a time-bounded processing environment suitable for clinical imaging workflows.

In some embodiments, the recognition subsystem architecture may be integrated with existing acquisition, reconstruction, and clinical data-handling pipelines without requiring replacement of preexisting scanner or hospital infrastructure. For example, the recognition voxel noise reduction module (1300) may be implemented as a post-reconstruction stage that operates on reconstructed images generated by an existing reconstruction workflow, including filtered back projection, ordered-subset expectation maximization, sensitivity encoding, generalized autocalibrating partially parallel acquisition, or another preexisting reconstruction pipeline. Likewise, the recognition adaptive exposure control module (1100), the recognition k-space reconstruction module (1200), the recognition voxel noise reduction module (1300), and the recognition diagnostic feature extraction module (1400) may interface with scanner hardware, controller pathways, reconstruction pathways, storage interfaces, reporting systems, or operator-facing systems through documented or otherwise supported interfaces. In some implementations, data may be received through a vendor interface (150), image objects and associated outputs may be written through DICOM egress (230), and the resulting images, enhanced images, feature maps, and associated metadata may be stored via storage or PACS interfaces (1700) or other storage systems. In this regard, the subsystem architecture of FIG. 10 (1000) may be deployed as an additional recognition-processing layer that remains compatible with the broader data-ingress, reconstruction, output, and logging structure illustrated in FIG. 1 (100) and FIG. 2 (200).

In some implementations, one or more portions of the recognition subsystem architecture may be deployed as software, firmware, programmable logic, microcode, containerized services, virtualized services, or combinations thereof. For example, the recognition subsystem architecture may be deployed as a software upgrade installed on existing compute hardware, as a sidecar compute appliance coupled to an imaging system, as a partially embedded module in which selected recognition computations are implemented in programmable logic, or as a hybrid deployment in which acquisition-side computations are performed in a lower-latency computing resource and reconstruction-side computations are performed in a higher-throughput computing resource. In some embodiments, the recognition subsystem architecture may be installed on newly manufactured systems or retrofitted to existing installed systems without requiring mechanical modification of scanner hardware. In such embodiments, the recognition subsystem architecture may preserve existing manufacturer interlocks, bounds, and other safety controls while extending the functionality of the imaging platform through software-controlled recognition processing. Accordingly, FIG. 10 (1000) may be understood as representing a recognition-processing architecture that is deployable across new and existing imaging platforms using multiple implementation pathways.

In some embodiments, the recognition subsystem architecture may generate, store, transmit, or display one or more outputs together with associated metadata that support traceability, quality assurance, regulatory review, or clinical workflow integration. For example, one or more of the reconstructed image output (1210), the enhanced image output (1310), the final image output (1510), and the diagnostic feature map output (1410) may be stored, transmitted, or presented together with metadata identifying acquisition settings, recognition coverage summaries, reconstruction settings, feature-extraction settings, timestamps, confidence indicators, or operator interactions. In some implementations, controller decisions and coverage summaries may be recorded in audit logs (240), image outputs may be written through DICOM egress (230), and operator status, protocol options, recognition-processing indicators, or override information may be presented through an operator console (170) or operator console outputs (1710). In some embodiments, calibration, initialization, and failover information may likewise be recorded so that recognition-based processing remains reviewable on a per-scan, per-protocol, or per-system basis. Thus, the computing and deployment implementations described herein may support not only execution of the recognition subsystem architecture, but also integration of its outputs and metadata into a clinical or technical environment that values reproducibility, traceability, and bounded operation.

In some implementations, the respective modules of the recognition subsystem architecture may share a common recognition coverage formalism while being independently mapped to different hardware resources, updated under different software release cycles, or enabled selectively according to protocol, modality, site policy, or clinical objective. For example, a first deployment may enable the recognition adaptive exposure control module (1100) and the recognition k-space reconstruction module (1200) while omitting the recognition voxel noise reduction module (1300) and the recognition diagnostic feature extraction module (1400), whereas a second deployment may enable all illustrated modules and may additionally store feature-map outputs and associated metadata for later review. Likewise, one or more modules may be activated only for selected protocols or only when sufficient compute resources are available. Accordingly, the computing and deployment implementations described in this section should be understood as encompassing fully integrated, partially integrated, software-only, firmware-assisted, and hardware-accelerated embodiments of the recognition subsystem architecture shown in FIG. 10 (1000).

What is claimed is:

1. A method for medical imaging, comprising:
receiving raw acquisition data and scanner or patient metadata from an imaging device;
computing a recognition coverage value for a region of interest from a quality signal r using a recognition coverage function Fcov parameterized by a recognition scaling parameter Xopt;
adaptively selecting or modulating one or more acquisition parameters during an exam based on the recognition coverage value;
reconstructing an image using the same recognition coverage value to weight at least one of frequency-domain filters, spatially varying regularization, and voxel-wise operations; and
outputting a medical image with associated metadata that records the acquisition parameters, the recognition coverage value, and reconstruction settings.

2. The method of claim 1 wherein the imaging device is at least one of computed tomography magnetic resonance imaging ultrasound positron emission tomography or digital radiography, wherein the acquisition parameters comprise at least one of exposure tube current tube voltage sequence timing echo time repetition time flip angle k-space sampling density beamforming weights time-gain compensation dwell time or count weighting, and wherein the reconstruction comprises at least one of frequency-domain filtering iterative inversion with spatially varying penalties or voxel-wise post-weighting.

3. The method of claim 1 wherein Fcov is a bounded monotone function of r that increases with coherent signal and decreases with noise or uncertainty, wherein in certain embodiments Fcov is computed as r divided by r plus Xopt, and wherein Xopt is in certain embodiments set to phi divided by pi and in other embodiments determined by optimizing an information criterion or fitting to calibration data.

4. The method of claim 1 wherein the quality signal r comprises at least one of a local signal-to-noise ratio a contrast or gradient-energy measure a residual from a forward model a detector health indicator a motion or physiological phase indicator or a count statistic, and wherein multiple quality signals are combined before applying the recognition coverage function.

5. The method of claim 1 wherein recognition coverage is computed per projection per k-space tile per receive channel per time bin and or per voxel, wherein the recognition coverage is smoothed or gated to ensure controller stability within a latency budget, and wherein rate limits and safety bounds constrain parameter updates.

6. The method of claim 1 wherein computed tomography is operated with view-wise tube-current modulation driven by recognition coverage with tube voltage optionally constrained to a protocol preset, and wherein reconstruction applies recognition-coverage-weighted frequency filtering and spatial penalties to reduce dose while preserving resolution and contrast-to-noise.

7. The method of claim 1 wherein magnetic resonance imaging is operated with coverage-guided selection of repetition time echo time flip angle and sampling density within vendor-approved bounds, and wherein reconstruction uses recognition-coverage-weighted parallel imaging compressed sensing or iterative inversion to harmonize sampling and regularization.

8. The method of claim 1 wherein ultrasound is operated with coverage-guided beamforming and time-gain compensation under device limits, and wherein post-processing applies recognition-coverage-weighted speckle suppression while preserving edges.

9. The method of claim 1 wherein positron emission tomography is operated with coverage-guided dwell time or count weighting across bed positions under a total scan-time budget, and wherein iterative reconstruction uses recognition-coverage-scaled priors to stabilize low-count regions.

10. The method of claim 1, further comprising:
applying a recognition voxel noise reduction operation to a reconstructed image by estimating, for one or more voxels, pixels, patches, neighborhoods, or regions of the reconstructed image, a local signal-to-noise ratio or a local confidence measure;
applying the recognition coverage function to the local signal-to-noise ratio or the local confidence measure; and
generating an enhanced image in which image regions associated with lower local signal-to-noise ratio or lower local confidence are attenuated relative to image regions associated with higher local signal-to-noise ratio or higher local confidence.

11. A medical imaging system, comprising:
scanner or detector acquisition hardware configured to acquire raw image data and scanner or patient metadata;
a recognition adaptive exposure control module configured to compute a recognition coverage value from a quality signal r using a recognition coverage function Fcov parameterized by a recognition scaling parameter Xopt, and to generate an exposure or acquisition command that adapts one or more acquisition parameters during an exam;
a recognition k-space reconstruction module configured to receive the raw image data and to generate a reconstructed image using the recognition coverage value to weight at least one of frequency-domain filters, spatially varying regularization, and voxel-wise operations;
a recognition voxel noise reduction module configured to receive the reconstructed image and to generate an enhanced image using a local signal-to-noise ratio or a local confidence measure weighted by the recognition coverage function;
a recognition diagnostic feature extraction module configured to receive a final image representation derived from the reconstructed image or the enhanced image, and to generate a diagnostic feature map using a tissue-contrast-based recognition coverage weighting; and
an interface configured to export at least one of the reconstructed image, the enhanced image, and the diagnostic feature map together with metadata that records the acquisition parameters, the recognition coverage value, and reconstruction settings.

12. The system of claim 11, wherein the scanner or detector acquisition hardware comprises at least one of an X-ray tube and detector, a set of radio-frequency coils and gradients, a transducer array, and a scintillation detector array, wherein the recognition adaptive exposure control module, the recognition k-space reconstruction module, the recognition voxel noise reduction module, and the recognition diagnostic feature extraction module execute on at least one of a central processing unit, a graphics processing unit, and a field-programmable gate array within a modality-specific latency budget, and wherein a vendor interface applies parameter updates within device constraints.

13. The system of claim 11, wherein Fcov is a bounded monotone function of r that increases with coherent signal and decreases with noise or uncertainty, wherein, in certain embodiments, Fcov is computed as r divided by r plus Xopt, and wherein Xopt is, in certain embodiments, set to phi divided by pi and, in other embodiments, determined by optimizing an information criterion or fitting to calibration data.

14. The system of claim 11, wherein the recognition adaptive exposure control module computes recognition coverage per projection, per k-space tile, per receive channel, per time bin, and/or per voxel with optional smoothing or gating for stability, and wherein the recognition k-space reconstruction module aligns the recognition coverage to corresponding spatial or temporal supports used during reconstruction.

15. A non-transitory computer-readable medium storing instructions that, when executed by one or more processors, cause the one or more processors to:
receive raw acquisition data and scanner or patient metadata from an imaging device;
compute a recognition coverage value from a quality signal r using a recognition coverage function Fcov parameterized by a recognition scaling parameter Xopt;
adapt one or more acquisition parameters during an exam based on the recognition coverage value;
reconstruct an image using the same recognition coverage value to weight at least one of frequency-domain filters, spatially varying regularization, and voxel-wise operations; and
output a medical image with associated metadata that records the acquisition parameters, the recognition coverage value, and reconstruction settings.

16. The method of claim 1, wherein recognition coverage is recorded as a map aligned to projections, k-space tiles, time bins, or voxels, wherein interpolation between supports is conservative and confidence-tracked, and wherein the same recognition coverage map used upstream is consumed downstream to reduce inconsistency between acquisition and reconstruction.

17. The method of claim 1 wherein parameter updates are subject to manufacturer interlocks and clinical policy limits, wherein operator overrides are permitted within bounds and recorded, and wherein on detection of out-of-range inputs the controller returns to a protocol baseline.

18. The system of claim 11, wherein the recognition diagnostic feature extraction module is configured to:
compute a tissue contrast metric for one or more voxels or image regions from at least one of a Hounsfield-unit deviation relative to a reference tissue, a T1 ratio, a T2 ratio, a deviation from atlas-based tissue values, a standardized uptake value ratio, an echogenicity ratio, and a B-mode intensity ratio;
apply the recognition coverage function to the tissue contrast metric; and
generate the diagnostic feature map from the tissue contrast metric and the applied recognition coverage function.

19. The system of claim 11, wherein the final image representation is generated by combining the reconstructed image and the enhanced image at a final image merge node using selection, averaging, weighted averaging, or blending before the recognition diagnostic feature extraction module generates the diagnostic feature map.

20. The non-transitory computer-readable medium of claim 15 wherein the instructions further cause the processors to determine Xopt by one of setting Xopt to phi divided by pi optimizing an information criterion over recent measurements or fitting to calibration data with hysteresis to avoid oscillation, and to constrain recognition-coverage-driven updates by rate limits and safety bounds to ensure stable operation.
